(19) United States
(12) Patent Application Publication
(10) Pub. No.: US 2021/0402204 A1
(43) Pub. Date: Dec. 30, 2021

(54) Breast Care Device

(71) Applicants: Rahul R. Rao, Phoenix, AZ (US); Ruby A. Obeng, Tempe, AZ (US); Alexandria Castillo, Tempe, AZ (US); Priscilla Han, Tempe, AZ (US); Katie Wilkinson, Gilbert, AZ (US); Kristin Huber, Tempe, AZ (US); Haley Ellis, Tucson, AZ (US)

(72) Inventors: Rahul R. Rao, Phoenix, AZ (US); Ruby A. Obeng, Tempe, AZ (US); Alexandria Castillo, Tempe, AZ (US); Priscilla Han, Tempe, AZ (US); Katie Wilkinson, Gilbert, AZ (US); Kristin Huber, Tempe, AZ (US); Haley Ellis, Tucson, AZ (US)

(73) Assignee: Desert Platforms, LLC, Phoenix, AZ (US)

(21) Appl. No.: 17/105,153

(22) Filed: Nov. 25, 2020

Related U.S. Application Data
(60) Provisional application No. 62/940,051, filed on Nov. 25, 2019.

(51) Int. Cl. A61N 5/06; A61M 35/00; A61H 23/02; A61H 15/00; A61N 7/00; A61B 8/08; A61N 1/36; A41C 3/04

(52) U.S. Cl. CPC A61N 5/0613; A61H 2205/082; A61H 23/02; A61H 15/0078; A61N 7/00; A61B 8/0825; A61N 1/36021; A41C 3/04; A61N 2005/0645; A61N 2005/0626; A61N 2005/0652; A61M 2210/1007; A61M 2205/3553; A61N 2005/0649; A61H 2015/0014; A61H 2201/10; A61H 2201/0207; A61H 2201/165; A61M 35/10

(57) ABSTRACT

A therapeutic device is placed over the breast and improves the quality of breastfeeding experience, aiming to heal and treat specific ailments nursing mothers encounter. The breast care device allows the user to maintain treatment while allowing for mobility during therapy. One aspect of the breast care device is made to contain and distribute therapeutic fluids, which are known to soothe breast pain, nipple cracks, anatomical variations, thrush, bites, milk blister, and other ailments. Another aspect of the breast care device contains an array of light elements for the purpose of light therapy, aiming to soothe the same ailments. The final aspect of the breast care device can contain a variety of electrical components, such as rollers for massage, vibration mechanisms for therapy, or ultrasound for imaging or therapy.

### CROSS REFERENCE TO RELATED APPLICATION

This application claims the benefit of U.S. Provisional Patent Application Ser. No. 62/940,051, filed Nov. 25, 2019.

### BACKGROUND OF THE INVENTION

### Field of Invention

The invention relates to a breast care device that addresses breast complications associated with breastfeeding which may include thrush or yeast infection, cracked nipples, breast pain, sore nipples, engorged breasts, clogged ducts, milk blisters, bites among others.

### Background

Many mothers experience different types of breast pain and discomfort during postpartum lactation. These conditions may include thrush or yeast infection, cracked nipples, breast pain, sore nipples, engorged breasts, clogged ducts, bites, or milk blisters. These complications can make breastfeeding an uncomfortable experience and even cause a mother to stop breastfeeding altogether.

Breastfeeding is a crucial part of a baby's development. Breast milk has been proven to protect babies from allergies and eczema, as well as reduce risk of viruses, urinary tract infections, and respiratory infections. Research has also suggested that babies who are breastfed have higher IQ scores later in their life in comparison to children who were not breastfed. In addition to benefitting the baby, breastfeeding is also beneficial for moms. A stronger bond is created between the mom and baby because of the skin-to-skin contact during the process. Also, breastfeeding helps lower the risk of breast and ovarian cancer and can help a mother in losing pregnancy weight. Although there are many benefits for breastfeeding, moms are still very likely to stop due to breastfeeding complications or pains.

To deal with these complications such as cracked nipples, thrush, bites, milk blisters among others, one helpful approach is for nursing mothers to treat the breast with a therapeutic solution such as saline solution after breastfeeding. Although the saline solution is helpful in treating breastfeeding pains, it can be difficult for the mother to apply the liquid to the breast.

In addition, current products treating breastfeeding complications use thermal therapy by providing heating or cooling to the breast but do not simultaneously provide medicated treatment or other therapy.

### SUMMARY OF THE INVENTION

The disclosed device is a breast care device that addresses breast complications associated with breastfeeding which includes thrush or yeast infection, cracked nipples, breast pain, sore nipples, engorged breasts, clogged ducts among others. The breast care device can be used independently or together with other treatment alternatives depending on the need of the use. The breast care device may be a complete bra, a strapless bra, or an insert to place into the user's bra. The disclosed device may have a system that can fit breasts of 95% female anthropometric data.

In some embodiments, the breast care device features a system to hold fluid or medication on the breast. The system may include a reservoir chamber that may be made out of a range of synthetic materials such as silicone, polypropylene, urethanes, and rubbers. ‘Reservoir chamber’ and ‘dome-like’ are synonyms with reservoir chamber used in embodiments where fluid is present and dome-like, describing the shape of the geometry, used when fluid is not present. This reservoir chamber may be able to contain fluid such as a therapeutic fluid inside against the breast. A breast care device may comprise an inlet port for receiving the therapeutic fluid and an outlet port to drain the therapeutic fluid. A therapeutic fluid may be a medication.

The reservoir chamber of an exemplary breast care device may include a seal to place and hold on to the breast. The seal will also prevent any fluid or medication from leaking out of the breast care device. The seal may be around the circumference of the reservoir chamber. The seal may be a plurality of passive seals that can hold the reservoir chamber to the breast. Passive seals may be aided with the use of adhesives to the body. The seal may also be an O-ring type design. The seal may be a vacuum seal that can include a syringe or a tube for creating negative pressure in order to improve the seal or fit to the body.

An exemplary breast care device may have an inlet port that can be used to introduce a therapeutic fluid into the reservoir chamber. The system may feature an inlet port that can introduce a therapeutic fluid through the use of a syringe. The system may feature a drain. The drain may be manual and the user will have the ability to empty the reservoir chamber themselves by opening the drain and closing it when needing to refill said reservoir chamber again. The drain may be automatic and empty the reservoir chamber out after a specific amount of time in order to decrease fluid contact with the breast. The automatic drain may drain the used therapeutic fluid to a compartment in the bottom portion of the reservoir chamber that acts as a second reservoir. The system may feature a medicated disposable insert of any shape that is placed inside the reservoir chamber. The disposable insert may be made out of foam. The disposable insert may be a medicated foam. The disposable insert may be a soaked gel pad. The disposable insert may be a pad soaked in therapeutic solution. The disposable insert may be a fluid pouch to release therapeutic fluid into the reservoir chamber and the reservoir chamber may have actuation feature to allow the user to release the fluid. The disposable insert or reservoir chamber may have channels to allow slow release of therapeutic fluid. The disposable insert may feature an adhesive to stick to the reservoir chamber.

According to another embodiment, the breast care device may feature said reservoir chamber as described in the previous embodiment that has therapeutic light elements attached to the inside part that may be facing the breast. The light elements may be light emitting diode light bulbs. The light elements may shine light on the breast for the purpose of phototherapy. Phototherapy treatment may be used to heal and relieve pain due to breastfeeding. The light elements may be powered by a battery pack. The light elements may be powered by wall power. The breast care device, similar to the previous embodiment, may also include said seal to the body that can be passive or a vacuum seal.

In another embodiment, the breast care device may feature said reservoir chamber as described in previous embodiments with an electrical grid at the device-breast interface. The electrical grid may be made of mesh fabric and may be used on its own without the reservoir chamber and placed on top of the breast. The electrified components may be powered by battery pack. The electrified components may be powered by an electrical outlet and power cord coupling the breast care device thereto. The breast care device, similar to the previous embodiments, may also include said seal to the body that may be passive or a vacuum seal. The electrified components may include a vibration mechanism. The electrified components may alternatively include a rolling mechanism for the purpose of massage.

The breast care device may have components of a smart system that enables a user to operate it using a phone or a remote control.

The breast care device may work in conjunction with a system defined as a “complete” bra, where the breast care device is sandwiched between the breast and the cup of the complete bra. For this usage, ‘complete’ can be described as an article of fabric clothing meant to cover and possibly offer support to the breast. A complete bra also can be described as having two straps over the shoulder that hold the fabric to the user.

Another embodiment of a complete bra would include a bra with one strap or without any straps.

The breast care device may have components including an ultrasound mechanism for the purpose of imaging or therapy. Such a component would be coupled to the device through either the reservoir chamber or attachment to the dome-like surface and may utilize a fluid-medium (within the reservoir chamber) for transmission of sound waves.

The summary of the invention is provided as a general introduction to some of the embodiments of the invention and is not intended to be limiting. Additional example embodiments including variations and alternative configurations of the invention are provided herein

### BRIEF DESCRIPTION OF THE SEVERAL VIEWS OF THE DRAWINGS

FIG. 1 is a front view showing the inside of an exemplary breast care device having a reservoir chamber, passive seals, an inlet port and a manual drain.

FIG. 2 is a side view of the exemplary breast care device shown in FIG. 1.

FIG. 3 is a perspective view of the exemplary breast care device shown in FIG. 1, being inserted into a bra cup.

FIG. 4 is a front view showing the inside of an alternate embodiment of the exemplary breast care device having a reservoir chamber, vacuum seal, an O-ring passive seal, an inlet port, and a manual drain.

FIG. 5 is a side view of the alternate embodiment of the exemplary breast care device shown in FIG. 4.

FIG. 6 is a front view showing the inside of an alternate embodiment of an exemplary breast care device having a reservoir chamber, passive seals, an inlet port, and an automatic drain.

FIG. 7 is a side view of the alternate embodiment of the exemplary breast care device shown in FIG. 6.

FIG. 8 is a perspective view of the alternate embodiment of the exemplary breast care device shown in FIG. 6, being inserted into a bra cup.

FIG. 9 is a back view showing the inside of an alternate embodiment of an exemplary breast care device having a reservoir chamber attached to a full bra, passive seals, an inlet port, and a manual drain.

FIG. 10 is a front view of the alternate embodiment of the exemplary breast care device shown in FIG. 9.

FIG. 11 is a front view of an alternate embodiment of the exemplary breast care device having a reservoir chamber attached to a strapless bra, vacuum seal, an inlet port, and a manual drain.

FIG. 12 is a back view showing the inside of the alternate embodiment of the exemplary breast care device shown in FIG. 11.

FIG. 13 is a perspective view of an alternate embodiment of an exemplary breast care device having a double walled reservoir chamber, passive seals, an inlet port, and a manual drain.

FIG. 14 is a perspective view of an alternate embodiment of the exemplary breast care device having a reservoir chamber with channels to allow slow release of fluids, an O-ring passive seal, an inlet port, and a disposable insert.

FIG. 15 is a front view showing the inside of an alternate embodiment of the exemplary breast care device having a reservoir chamber, passive seals, an inlet port, and a disposable insert.

FIG. 16 is a side view of the alternate embodiment of the exemplary breast care device shown in FIG. 15.

FIG. 17 is a front view showing the inside of an alternate embodiment of the exemplary breast care device having a reservoir chamber, passive seals, and the disposable insert is a pad soaked in therapeutic solution.

FIG. 18 is a front view showing the inside of an alternate embodiment of the exemplary breast care device having a reservoir chamber, passive seals, a disposable insert that is a fluid pouch, and a disposable insert made of foam.

FIG. 19 is a front view of a disposable insert with an adhesive that can be placed inside the reservoir chamber.

FIG. 20 is a back view of the disposable insert shown in FIG. 19 with the adhesive visible.

FIG. 21 is a front view of a disposable insert is an oversoaked gel pad that can be placed inside the reservoir chamber.

FIG. 22 is a back view of the disposable insert shown in FIG. 21 with the adhesive visible.

FIG. 23 is front view of a disposable insert that has channels to allow slow release of fluid and can be placed in the reservoir chamber.

FIG. 24 is a back view of the disposable insert shown in FIG. 23 with the adhesive visible.

FIG. 25 is a side view of an alternate embodiment of the exemplary breast care device having passive seals, an array of light elements, and holes for air flow.

FIG. 26 is a front view of an alternate embodiment of the exemplary breast care device having, passive seals, an array of light elements, and holes for air flow.

FIG. 27 is a front view showing the inside of an alternate embodiment of the exemplary breast care device having a dome-like shape, passive seals, and electrical components in a grid formation powered by a battery pack.

FIG. 28 is a side view of the of the exemplary breast care device shown in FIG. 27.

FIG. 29 is a front view showing the inside of an alternate embodiment of the exemplary breast care device having a dome-like shape, passive seals, and electrical components in a grid formation powered by wall power.

FIG. 30 is a side view of the of the exemplary breast care device shown in FIG. 29.

FIG. 31 is a front view showing the inside of an alternate embodiment of the exemplary breast care device having a dome-like shape, passive seals, a vacuum seal, and a vibration mechanism powered by a battery pack.

FIG. 32 is a side view of the alternate embodiment of the exemplary breast care device shown in FIG. 31.

FIG. 33 is a front view showing the inside of an alternate embodiment of the exemplary breast care device having a dome-like shape, passive seals, a vacuum seal, and a vibration mechanism powered by wall power.

FIG. 34 is a side view of the alternate embodiment of the exemplary breast care device shown in FIG. 33.

### DETAILED DESCRIPTION OF THE ILLUSTRATED EMBODIMENTS

The accompanying drawings are included to provide a further understanding of the invention and are incorporated in and constitute a part of this specification, illustrate embodiments of the invention, and together with the description serve to explain the principles of the invention.

Corresponding reference characters indicate corresponding parts throughout the several views of the figures. The figures represent an illustration of some of the embodiments of the present invention and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

Following is a list of elements corresponding to a particular element referred to herein:
10 Breast Care Device
100 Reservoir Chamber or Otherwise Dome-like Shape
101 Seal
102 Inlet Port
103 Drain
104 Light elements
105 Electrified Components
106 Vibration mechanism
107 Disposable insert
108 Adhesive
109 Power cord
110 Holes for Airflow

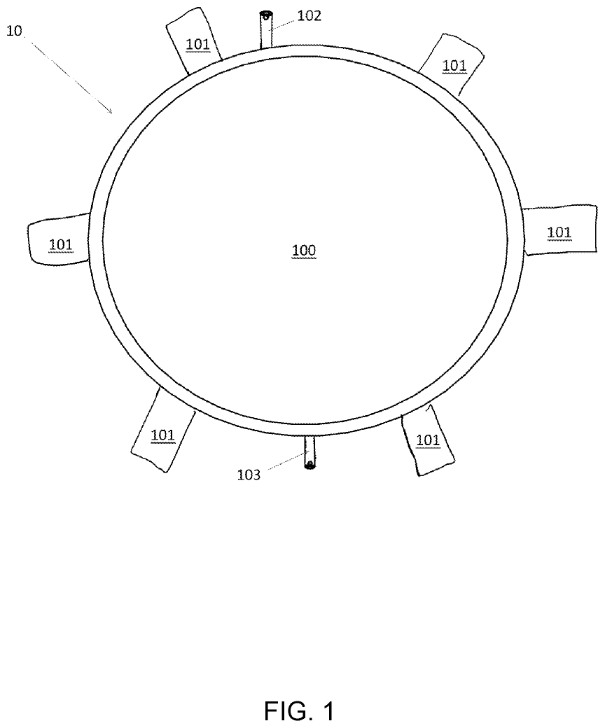

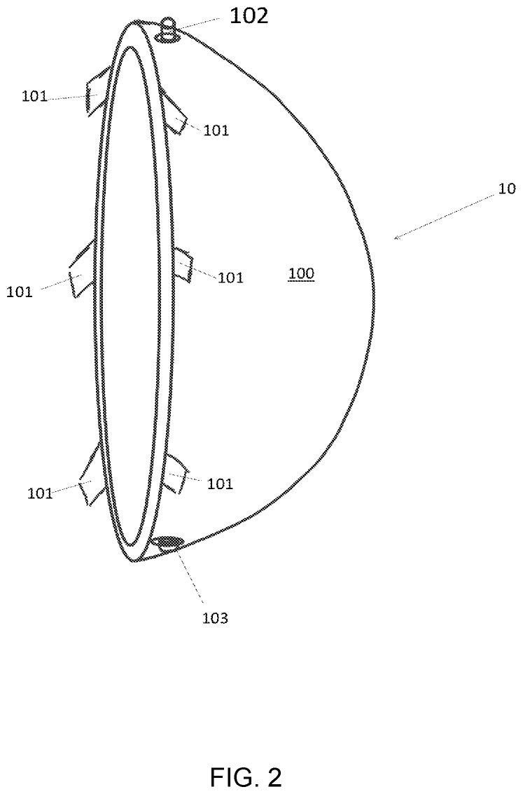

As shown in FIGS. 1 and 2, an exemplary breast care device 10 has a reservoir chamber 100, a plurality of passive seals 101 configured around the reservoir chamber, an inlet port 102 for receiving therapeutic fluid, and a drain 103 to release the fluid out of the chamber so the user may take off the device easily with respect to fluid management.

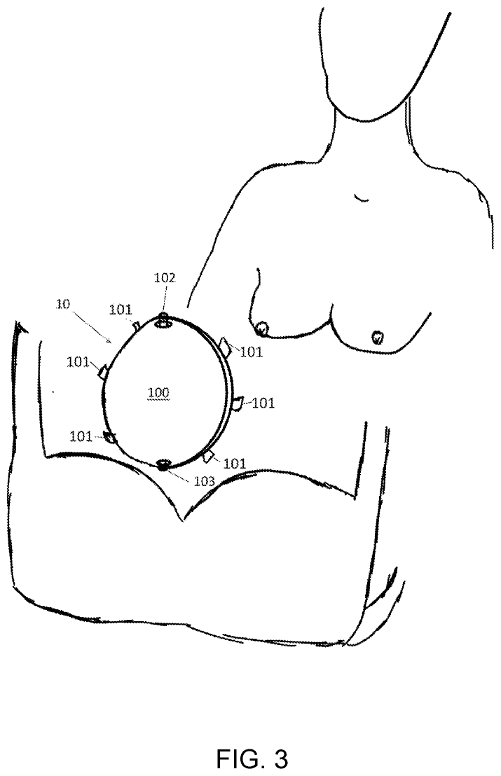

FIG. 3 shows a perspective view of the previous embodiment and how the breast care device 10 will be placed inside of the bra and on top of the breast.

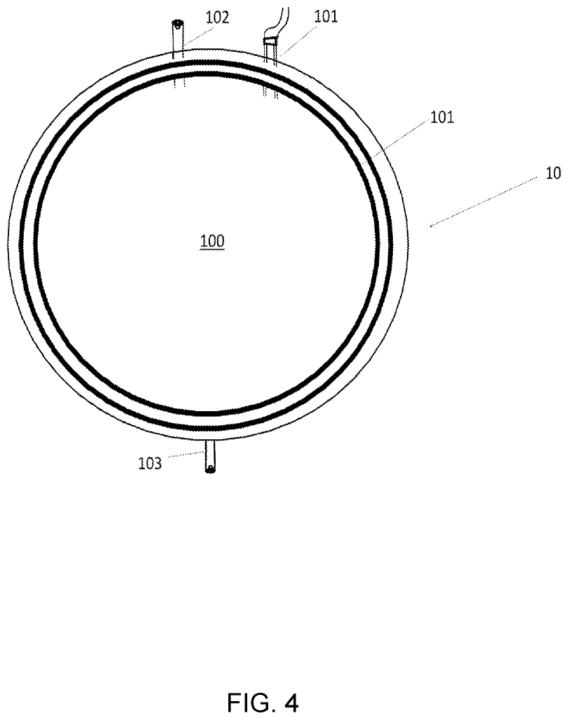

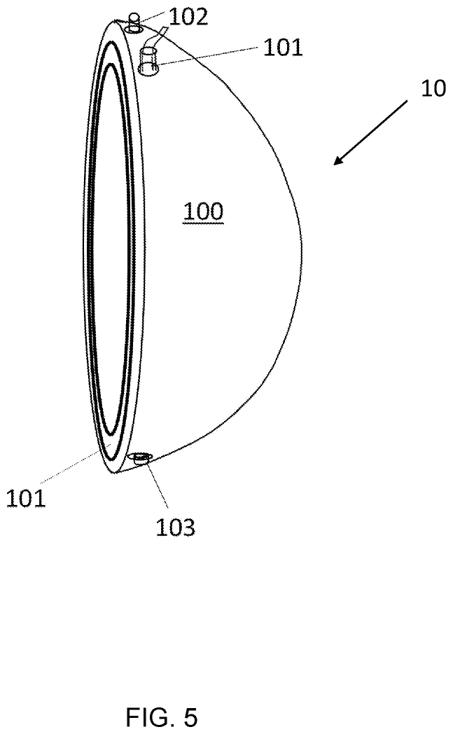

As shown in FIGS. 4 and 5, an exemplary breast care device 10 has a reservoir chamber 100, a vacuum seal 101 configured on the reservoir chamber, an O-ring passive seal 101, an inlet port 102 for receiving a therapeutic fluid, and a drain 103 to release the fluid out of the chamber so the user may take off the device easily with respect to fluid management.

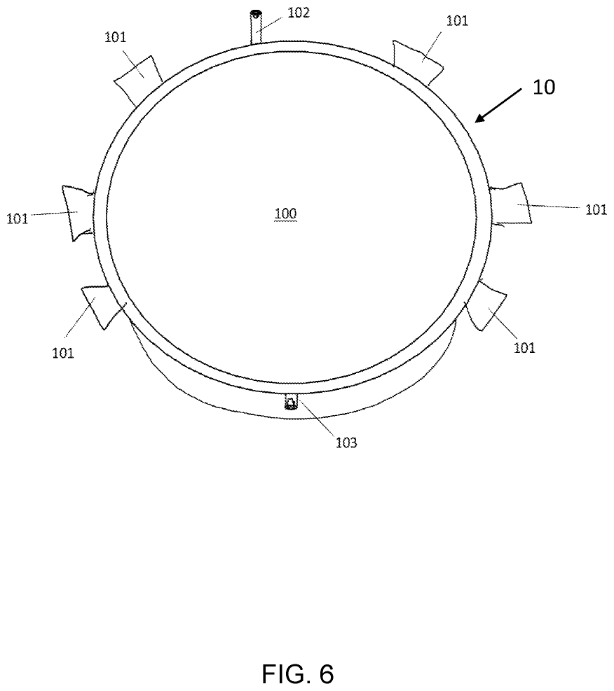

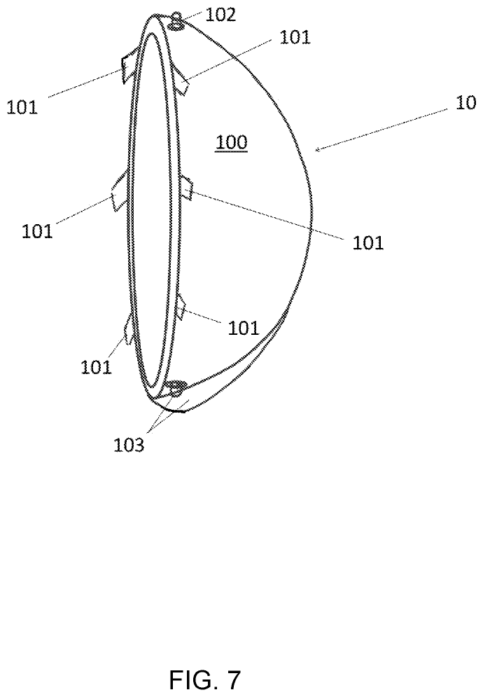

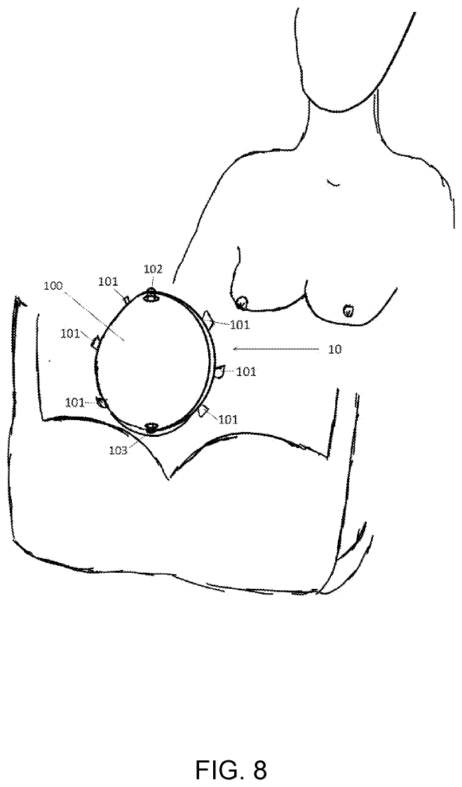

As shown in FIGS. 6, 7, and 8, an exemplary breast care device 10 has a reservoir chamber 100, a plurality of passive seals 101 configured around the reservoir chamber, an inlet port 102 for receiving a therapeutic fluid, and an automatic drain 103 to release the fluid out of the chamber after a specific amount of time into a lower chamber to decrease fluid contact with the breast.

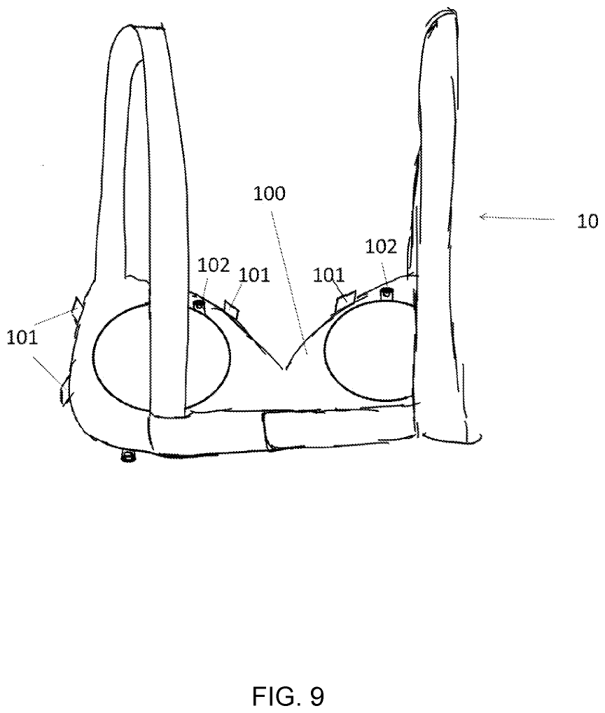

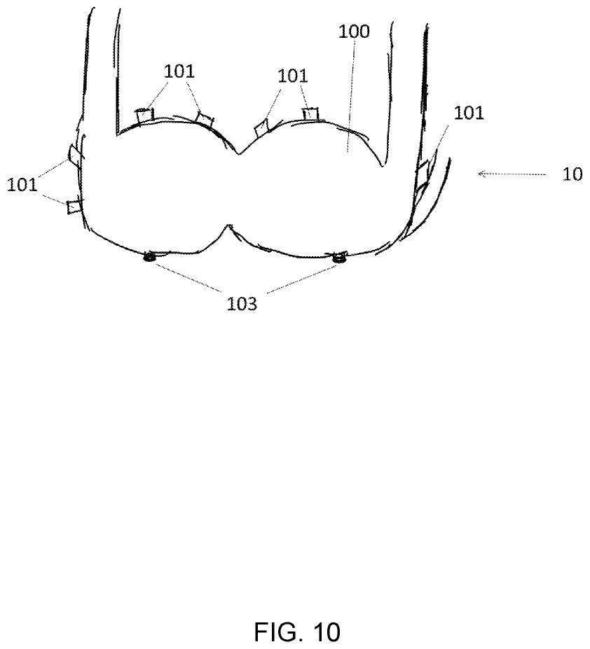

As shown in FIGS. 9 and 10, an exemplary breast care device 10 has a reservoir chamber 100 that is a part of a complete bra, a plurality of passive seals 101 configured around the reservoir chamber, multiple inlet ports 102 for receiving therapeutic fluid, and multiple drains 103 to release the fluid out of the chamber so the user may take off the device easily with respect to fluid management.

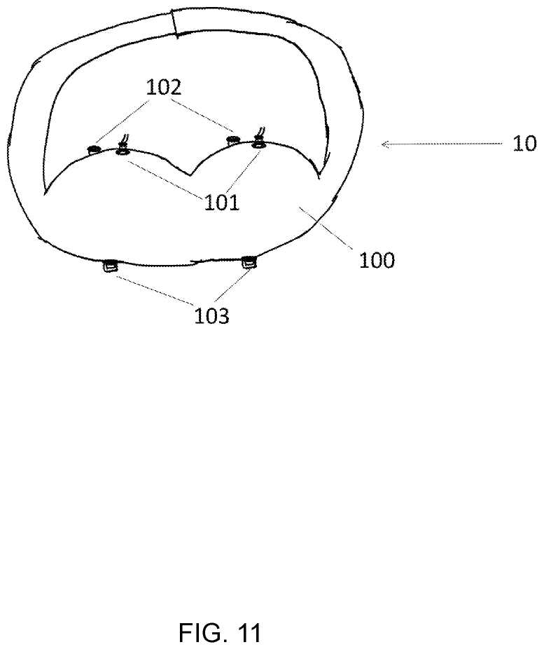

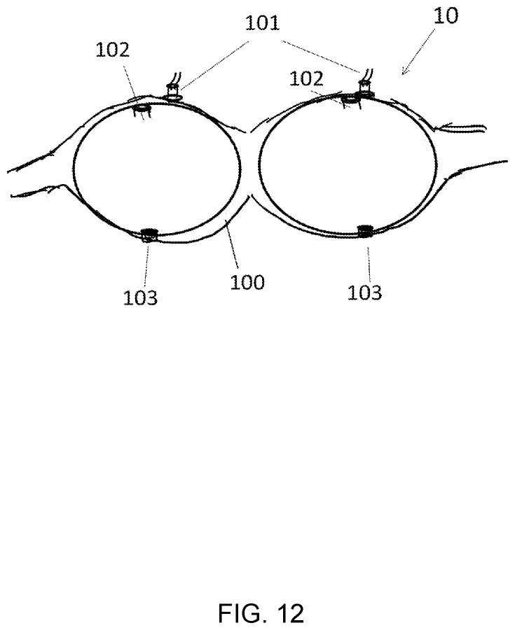

As shown in FIGS. 11 and 12, an exemplary breast care device 10 has a reservoir chamber 100 that is a part of a strapless bra, multiple vacuum seals 101 configured on the reservoir chamber, multiple inlet ports 102 for receiving therapeutic fluid, and multiple drains 103 to release the fluid out of the chamber so the user may take off the device easily with respect to fluid management.

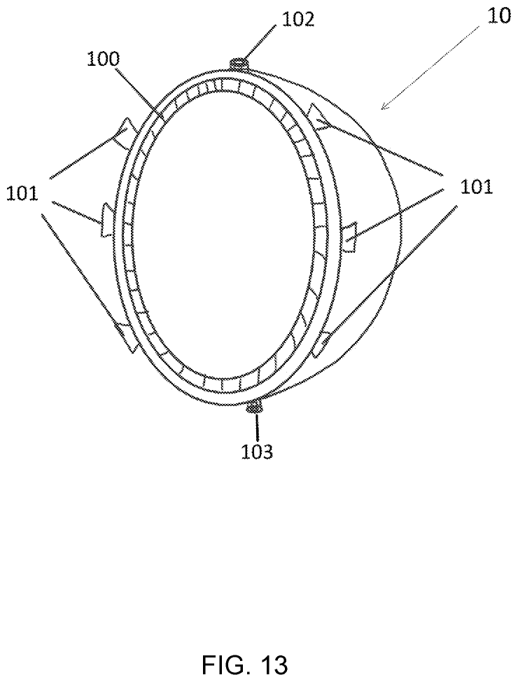

As shown in FIG. 13, an exemplary breast care device 10 has a double walled reservoir chamber 100, a plurality of passive seals 101 configured around the reservoir chamber, an inlet port 102 for receiving a therapeutic fluid, and a drain 103 to release the fluid out of the chamber so the user may take off the device easily with respect to fluid management.

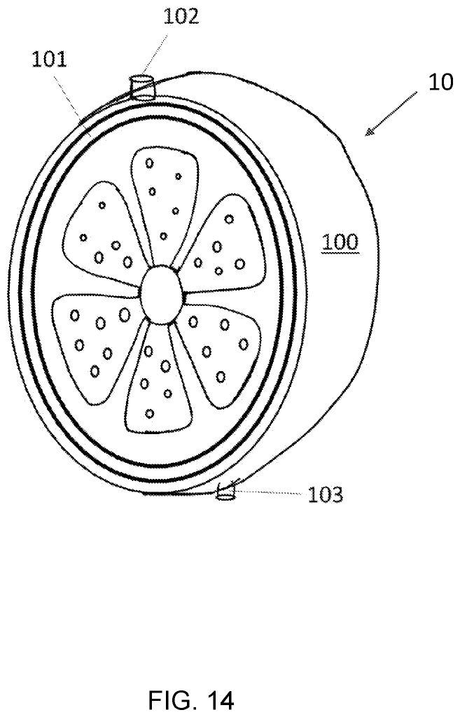

As shown in FIG. 14, an exemplary breast care device 10 has a reservoir chamber 100, an O-ring passive seal 101, an inlet port 102 where therapeutic fluid will be introduced to the top, the reservoir chamber 100 with channels that allow slow release of fluid, and a manual drain to release fluid out of the chamber so the user may take off the device easily with respect to fluid management.

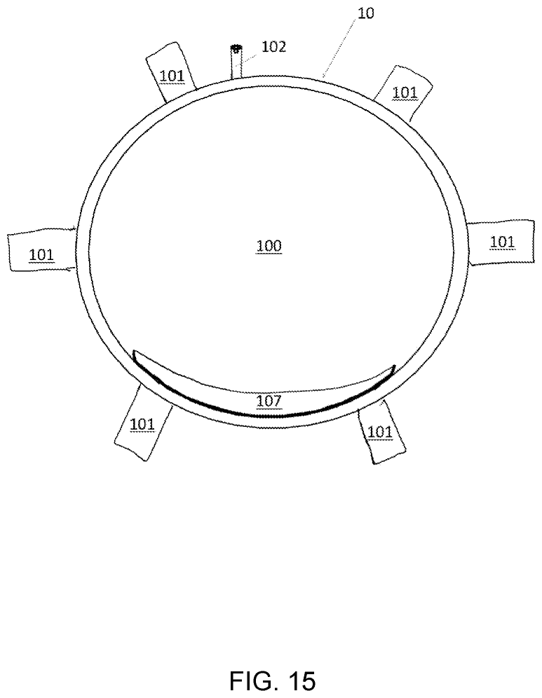

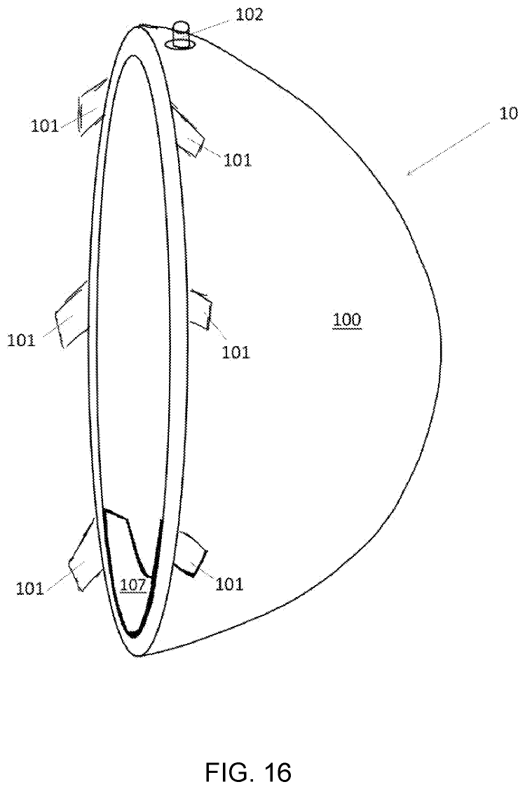

As shown in FIGS. 15 and 16, an exemplary breast care device 10 has a reservoir chamber 100, a plurality of passive seals 101 configured around the reservoir chamber, an inlet port 102 for receiving a therapeutic fluid, and a disposable insert 107 to soak up the therapeutic fluid in a controlled manner.

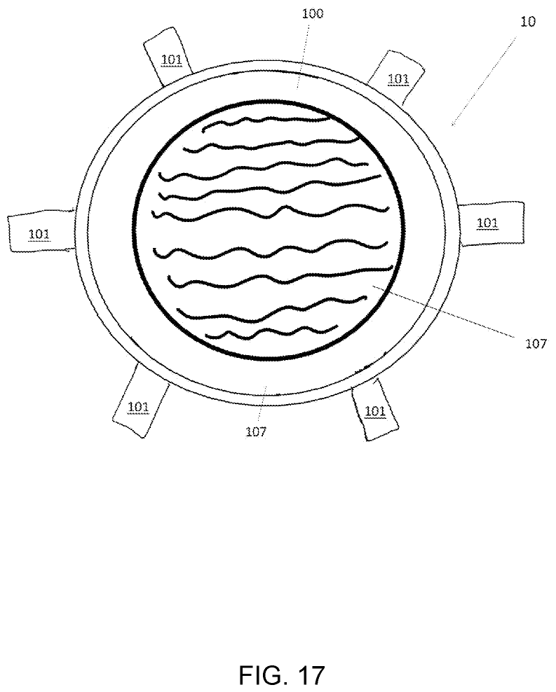

As shown in FIG. 17, an exemplary breast care device 10 has a reservoir chamber 100, a plurality of passive seals 101 configured around the reservoir chamber, and a disposable insert 107 that is a pad soaked in therapeutic solution to be placed on the breast.

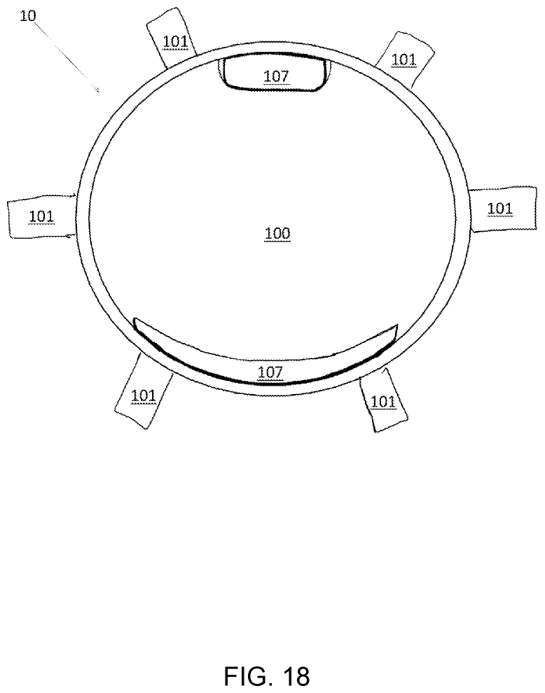

As shown in FIG. 18, an exemplary breast care device 10 has a reservoir chamber 100, a plurality of passive seals 101 configured around the reservoir chamber, a disposable insert 107 that is a fluid pouch filled with therapeutic fluid that can be released inside the reservoir chamber and another disposable insert 107 to soak up the therapeutic fluid in a controlled manner.

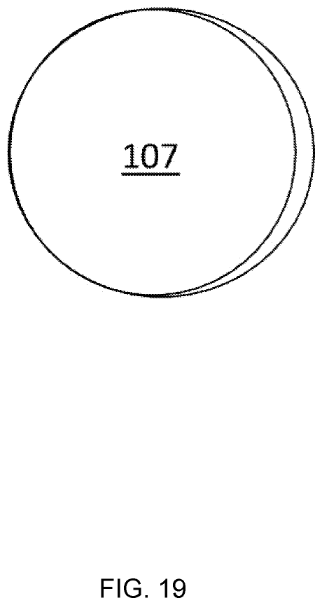

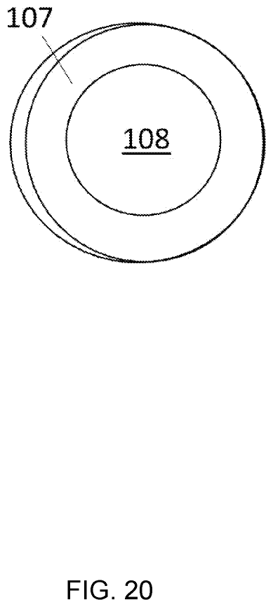

As shown in FIGS. 19 and 20, a disposable insert 107, may be placed inside the exemplary breast care device 10, and has an adhesive 108.

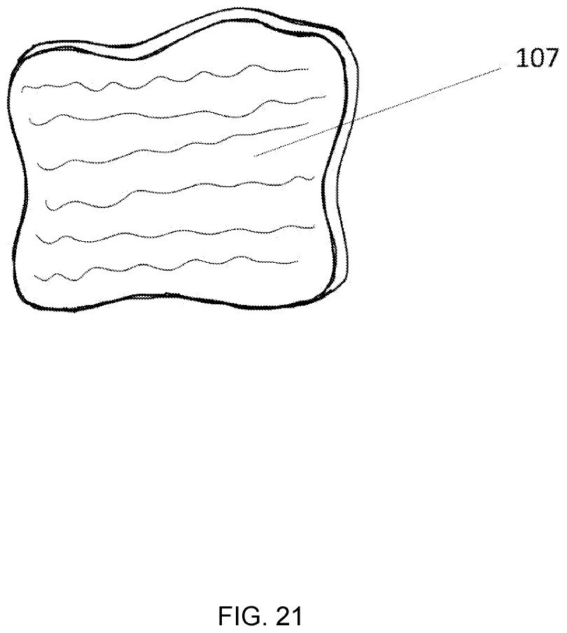

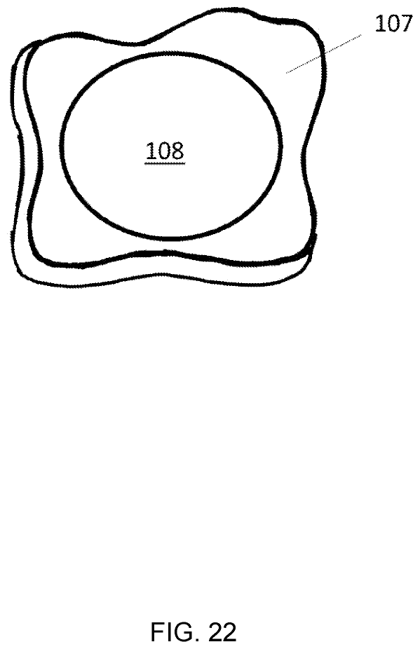

As shown in FIGS. 21 and 22, a disposable insert 107, that may be placed inside the exemplary breast care device 10, is an oversoaked gel pad with adhesive 108.

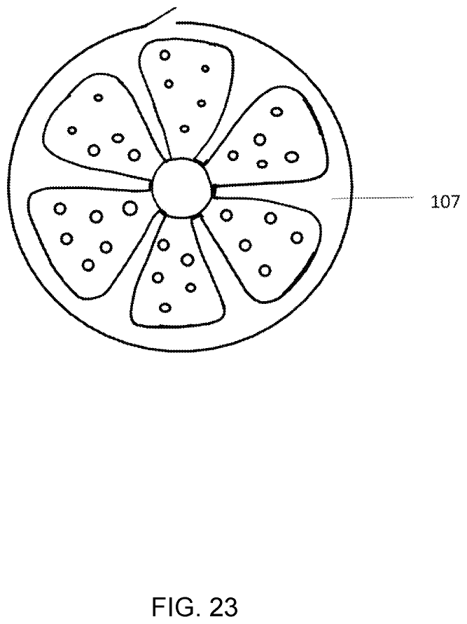

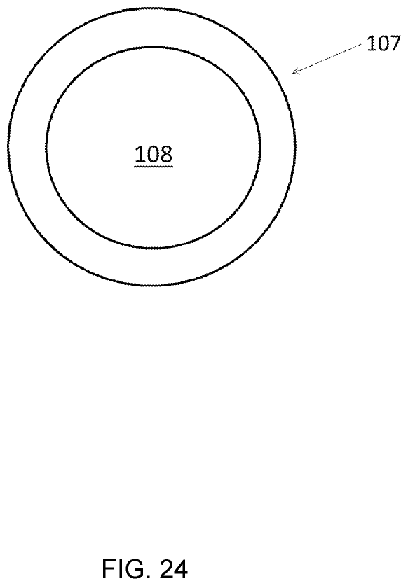

As shown in FIGS. 23 and 24, a disposable insert 107 that may be placed inside the exemplary breast care device 10, where therapeutic fluid will be introduced to the top and will have channels that will allow slow release of fluids with adhesive 108.

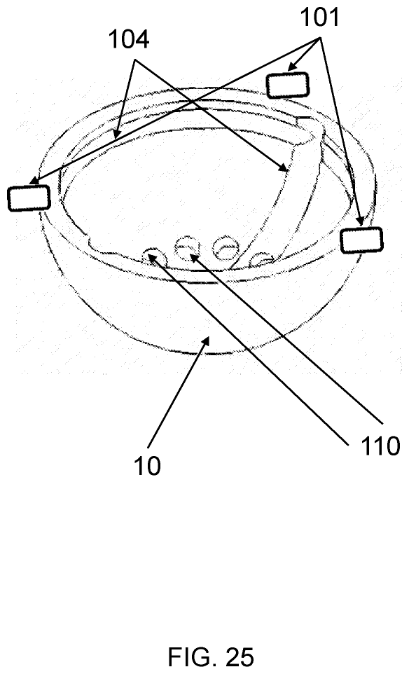

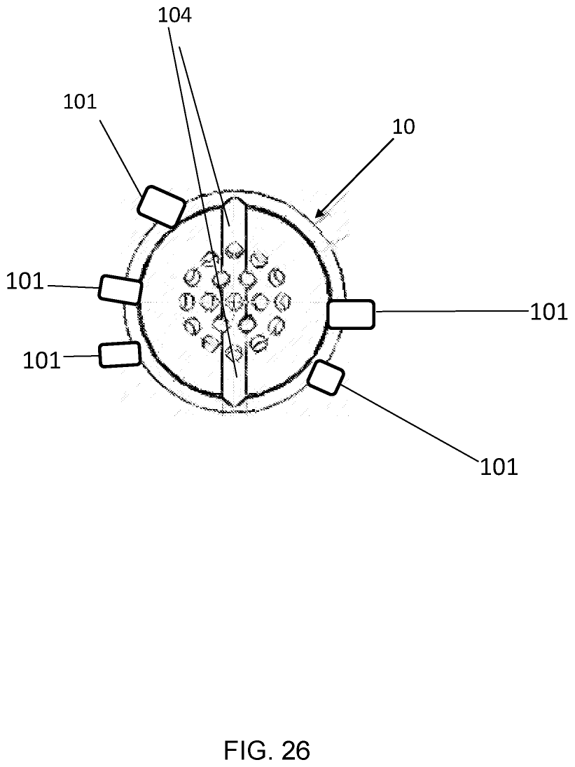

As shown in FIG. 25-26, an exemplary breast care device 10 has a reservoir chamber 100, a plurality of passive seals 101 configured around the dome-like shape, and an array of light elements 104 attached to the inside of the reservoir chamber 100 to shine therapeutic light towards the breast.

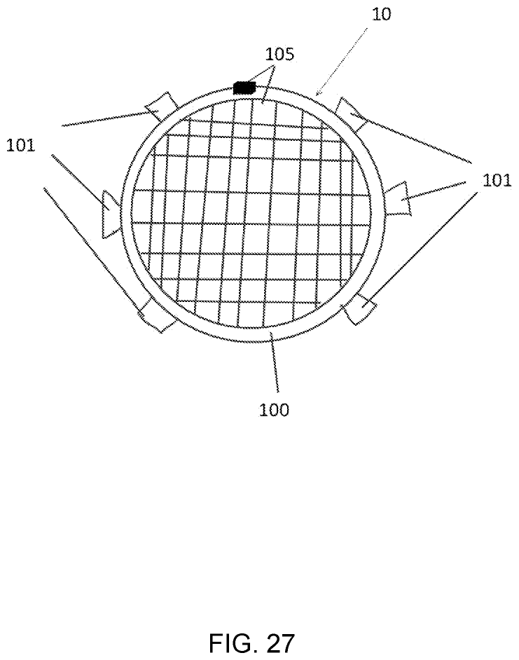

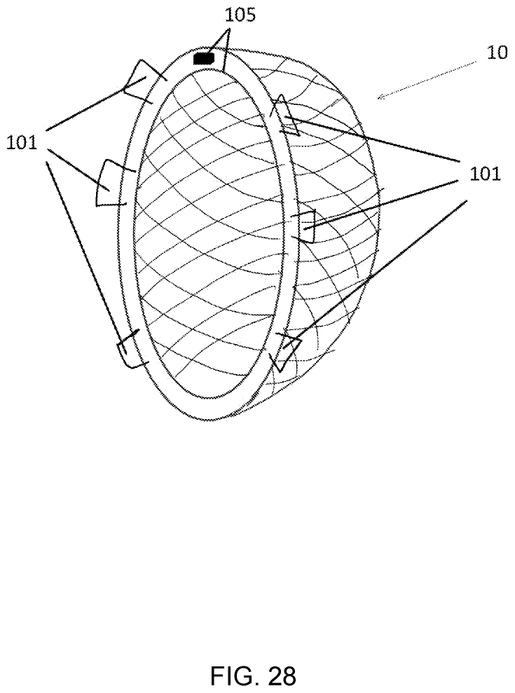

As shown in FIG. 27-28, an exemplary breast care device 10 has a dome-like shape 100, a plurality of passive seals 101 configured around the reservoir chamber, and electrified components 105 in a grid configuration with a mesh composition around the breast powered by a battery pack.

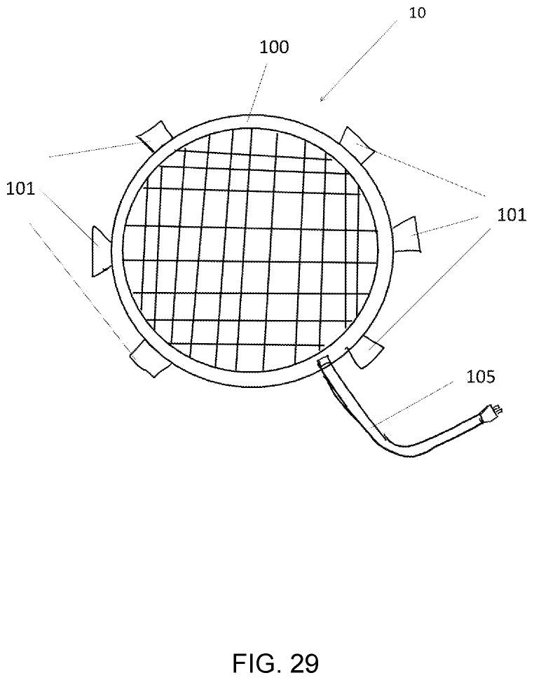

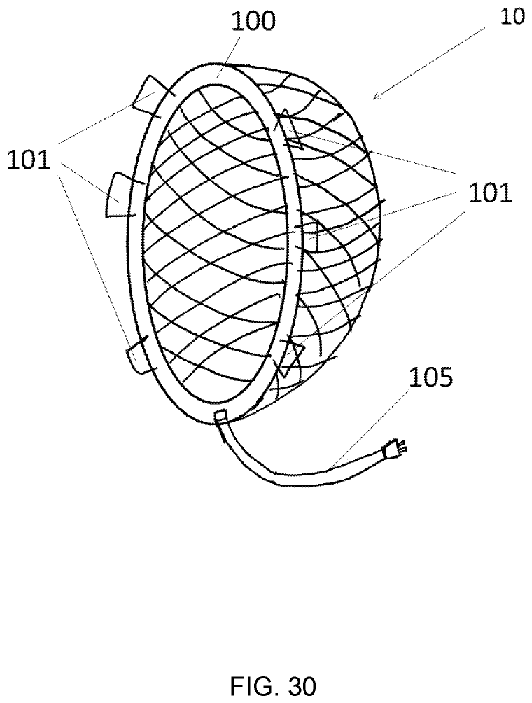

As shown in FIG. 29-30, an exemplary breast care device 10 has a dome-like shape 100, a plurality of passive seals 101 configured around the reservoir chamber, and electrified components 105 in a grid configuration with a mesh composition around the breast powered by wall power. A power cord 109 may extend from the electrified components 105 and be configured with a plug for connecting with a power source, such as an electrical outlet.

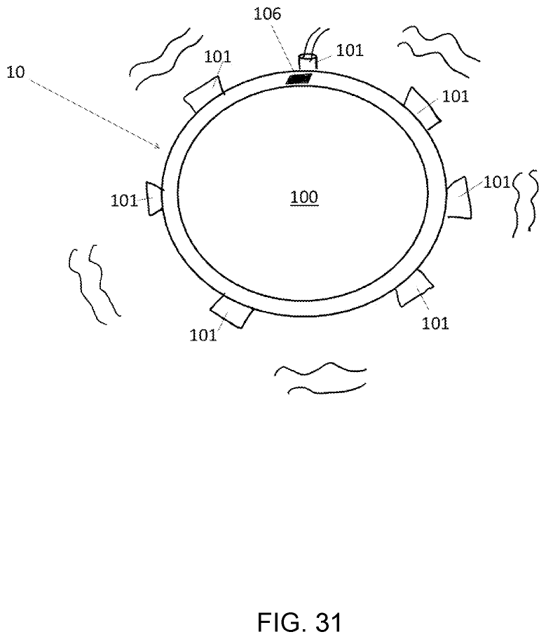

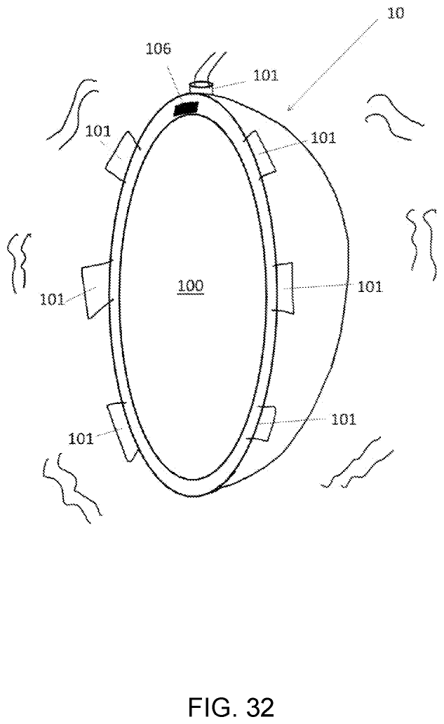

As shown in FIG. 31-32, an exemplary breast care device 10 has a reservoir chamber 100, a plurality of passive seals 101 configured around the dome-like shape, and a vibration mechanism 105 powered by a battery pack.

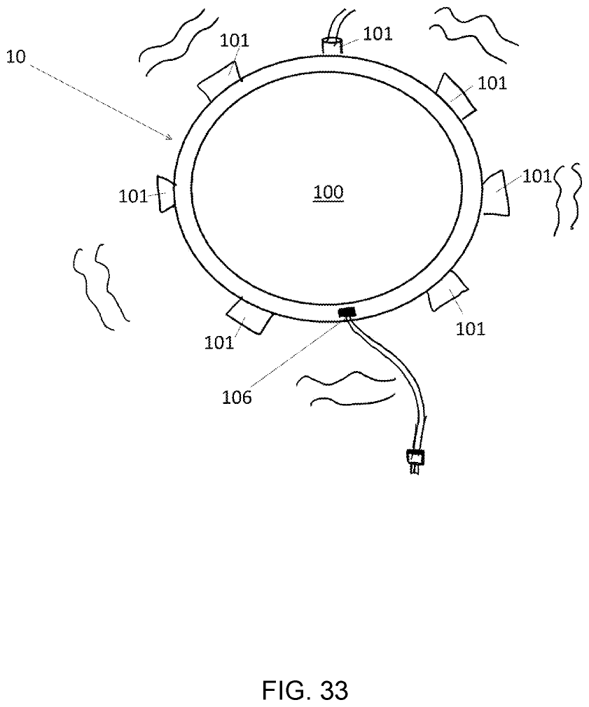

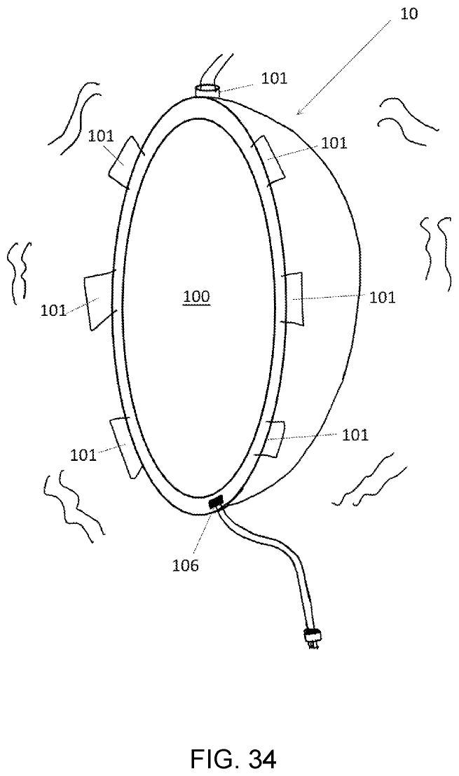

As shown in FIG. 33-34, an exemplary breast care device 10 has a dome-like shape 100, a plurality of passive seals 101 configured around the dome-like shape, and a vibration mechanism 105 coupled with a power cord 109 configured with a plug for connecting with a power source, such as an electrical outlet.

1. A medical device comprising:
a) a dome-like breast care device comprising:
i) an array of light elements;
ii) a seal to the breast.

2. The device of claim 1, wherein the array of light elements is comprising:
a) individual light source(s);
b) Light element strip(s).

3. The device of claim 2, wherein the seal is comprising:
a) A passive seal to the breast;

4. The device of claim 3, wherein the seal is comprising:
a) A vacuum seal to the breast.

5. The device of claim 3, wherein the device form is comprising:
a) Complete bra;

6. The device of claim 5, wherein the device form is comprising:
a) A complete bra that does not have straps.

7. The device of claim 3, wherein the device form is comprising:
a) A device that works in conjunction with a complete bra.

8. The device of claim 7, wherein the device form is comprising:
a) A rigid dome.

9. The device of claim 7, wherein the device form is comprising:
a) A flexible dome.

10. The device of claim 7, wherein the device is comprising:
a) Vents for natural airflow;
b) Forced air for artificial airflow;
c) heat source.

11. The device of claim 10, wherein the device comprises:
a) Incorporation of a smart device that can be operated remotely.

12. The device of claim 11, wherein the power source is comprising:
a) Rechargeable battery or;
b) Disposable battery or;
c) Corded power supply.

13. The device of claim 12, wherein the device comprises:
a) Reusable components or
b) Disposable components.

14. A medical device comprising:
a) a dome-like breast care device comprising:
i) An array of light sources within the dome for phototherapy;
ii) A passive seal to the breast on the perimeter of the dome;
iii) Works in conjunction and is kept in place on the breast by the complete bra;
iv) Vents within the dome for natural airflow;
v) Incorporation of a smart device that can be operated remotely;
vi) Rechargeable battery to power the device;
vii) Disposable seal components.

15. A medical device comprising:
a) a dome-like breast care device comprising:
i) a reservoir chamber, the said reservoir chamber comprising of:
ii) an inlet port to receive fluids;
iii) an outlet port to discharge fluids;
iv) a sealing device to form a sealed grip on to the breast
v) incorporation of a smart device that can be operated remotely

16. The device of claim 16, wherein the drain has an automatic feature and compartment for drainage to discontinue fluid contact with the breast.

17. The device of claim 16, wherein the device form is comprising:
a) Complete bra;
b) Doubled wall reservoir;
c) Geometries inhibiting flow rate for slow fluid release.

18. The device of claim 17, wherein the device form is comprising:
a) A complete bra that does not have straps.

19. The device of claim 15, wherein the device form is comprising:
a) A device that works in conjunction with a complete bra.

20. The device of claim 19, further comprising a separate component placed inside the reservoir chamber to effect medicated therapy comprising:
a) component is a pad soaked in solution;
b) component has an adhesive backing;

21. The device of claim 19 further comprising a separate component placed inside the reservoir chamber:
a) component is a fluid pouch to release fluid;
b) component has geometries for slow fluid release.

22. A medical device comprising:
a) a dome-like breast care device comprising:
i) a seal to the breast;
ii) electrified components around the breast
iii) a battery to power the device

23. The device of claim 22, wherein the electrified components comprise of:
a) Vibration mechanism;
b) Rolling mechanism for massage;

24. The system of claim 22, wherein the electrified components comprise of:
a) Ultrasound Imaging;
b) Ultrasound Therapy.

25. The system of claim 22, wherein the electrified components comprise of:
a) Transcutaneous electrical nerve stimulation (TENS).

26. The device of claim 24, where in the device is comprising:
a) Vents for natural airflow;
b) Forced air for artificial airflow;
c) heat source.

27. The device of claim 26, wherein the device form comprises of:
a) A complete bra;

28. The device of claim 27, wherein the device form is comprising:
a) A complete bra that does not have straps.

29. The device of claim 26, wherein the device form is comprising:
a) A device that works in conjunction with a complete bra.

30. The device of claim 29, wherein the device comprises:
a) Incorporation of a smart device that can be operated remotely.
